(19) United States
(12) Patent Application Publication
(10) Pub. No.: US 2007/0213701 A1
(43) Pub. Date: Sep. 13, 2007

(54) Implanting devices and methods thereof

(76) Inventor: Mark Kraft, Las Vegas, NV (US)

Correspondence Address: WEIDE & MILLER, LTD., 7251 W. LAKE MEAD BLVD., SUITE 530, LAS VEGAS, NV, 89128, US

(21) Appl. No.: 11/353,774

(22) Filed: Feb. 14, 2006

(51) Int. Cl. A61B 18/04

(52) U.S. Cl. 606/32

(57) ABSTRACT

An implantation device is disclosed, with the implantation device including an introducer member having an opening and a substantially flexible catheter located in an inner portion of the introducer member. The catheter selectively extends from the introducer member and is configured to both contain and selectively deliver an implantable member. The implantable member is guided by the catheter along a circumferential path of an inner portion of a body receiving treatment, and a blocker of the implantation device prevents retraction of the implantable member from the inner portion of the body when retracting the catheter into the introducer member after implanting the implantable member. When the body is an intervertebral disc, the catheter follows a substantially circumferential path located in a transition zone between the annulus fibrosis and the nucleus pulposus of the intervertebral disc and deposits the implantable member comprising medication, stem cell infusion, or both.

### FIELD OF THE INVENTION

This invention relates generally to devices and methods for treating a body and, more specifically, to improved devices for circumferential application of material into a portion of a body and methods for distributing therapeutic materials selectively into locations within the body.

### BACKGROUND OF THE INVENTION

In the past a number of devices and methods have been used to treat defective body organs and tissues of a patient. In the case of treatment of defective intervertebral discs, such as caused by herniation of vertebra or slipped discs, when the pain generated by the defective disc becomes chronic, surgical treatments are typically performed. These surgical treatments often result in destruction of at least portions of the defective disc to remove the interaction of the defective disc with surrounding nerve endings. Typically these surgical treatments (often percutaneous treatments) involve mechanical removal, chemical dissolution, electro-thermal, radio frequency electric currents, laser or ultrasound techniques to reduce the inner volume of the defective disc to reduce internal disc pressure thus reducing external pressure exerted on adjacent nerves. Some procedures also include total surgical removal of the defective disc (called discectomy), with replacement by appropriately shaped prosthesis.

Another alternative treatment is disc fusion wherein the vertebra connecting the defective disc are fused together to preserve the physical integrity of the patient's backbone in the region of the defective disc. Such treatments have various degrees of success, but can result in reduced mobility of the patient. Additionally, such treatments may result in damage to or a worsening of the patient's condition since surgery is complex to perform and uncertain in outcome. As can be appreciated, any form degree of worsening of the condition is a highly undesirable outcome.

The intervertebral disc is a complex structure comprising a central, gelatinous nucleus pulposus (nucleus) encircled by a tough, fibrous, semi-elastic annulus fibrosus (annulus). One function of the intervertebral disc is to serve as a shock absorber between the vertebrae. It is believed that the nucleus pulposus comprises an amorphous colloidal mass of gelatinous material containing glycosaminoglycans, collagen fibrils, mineral salt, water and cellular elements. The nucleus pulposus is normally under pressure and is contained within an ovoid cavity formed laterally by the annulus fibrosus and bounded by thin plates of hyaline (i.e., clear to translucent non-fibrous or non-granular material) cartilage endplates covering the adjacent vertebrae. Thin cartilage endplates and vertebral body ring apophyseal (i.e., naturally enlarged) attachments of the annulus fibrosus couple the disc to the vertebrae craniad (i.e., in the direction of the brain) and caudad (i.e. in the direction of the tail bone) to the disc. In a healthy disc the nucleus pulposus is gelatinous and somewhat fluid while the annulus fibrosus comprises circularly arranged fibers of concentric layers of fibrocartilage, in which collagen fibers are arranged in parallel strands running obliquely between vertebral bodies. The border between the nucleus pulposus and the annulus fibrosus is not distinct in a healthy adult disc. It is the border between the nucleus pulposus and the annulus fibrosus in the intervertebral disc comprises a gradual transition layer.

Since lower back injuries and chronic back pain are a major health problem which debilitates patients and results in expenditure of a large proportion of funds allocated for health care, social assistance, and disability programs, it is desirable to provide improved devices and methods for treating these conditions with better procedures. Indeed, it is preferable to treat such conditions without destroying any underlying tissue, which would thereby avoid postoperative surgery such as introduction of prosthesis. However, prior art methods suffer from being extremely invasive and destructive.

In other body organs, a similar situation applies, namely, the desire to accurately implant medically effective materials into discrete locations of the body organ. If the implantation is inaccurate, such medications would never reach their intended target and therefore be marginally effective at best and more likely be ineffective. However, with accurate delivery of medications by single use or continuous infusion of medications to specifically targeted portions of the body organ it would be possible to more effectively treat patient body organs. Prior art methods suffer from being undesirably invasive, or from poorly dispersing medications.

The current disclosure describes improved devices and methods for placement of material into or treatment of defective or injured intervertebral discs and other body organs to non-destructively treat the intervertebral discs and other body organs.

### SUMMARY OF THE INVENTION

To overcome the drawbacks of the prior art, an intervertebral disc implantation device is disclosed. In one embodiment the device comprises an introducer member having an opening and a substantially flexible catheter. The catheter is located in an inner portion of the introducer member and the catheter configured may be configured to selectively extend from the inner portion of the introducer member through the opening of the introducer member. The catheter may comprise a thermoplastic needle, an electrically deformable needle, or any other type of needle. The catheter may be configured to both contain and selectively deliver an implantable member such that the implantable member is guided by the catheter along a path of an inner portion of the intervertebral disc of a person. The path may be located in a transition zone between a nucleus pulposus and an annulus fibrosis of the intervertebral disc. The device may also be configured such that a portion of the intervertebral disc implantation device prevents retraction of the implantable member from the inner portion of the intervertebral disc when the catheter is retracted into the introducer member after implanting the implantable member.

In one embodiment the path has a substantially circumferential shape and, the implantable member is substantially located in a substantially circumferentially shaped portion of the transition zone or nucleus pulposis. In one configuration, the implantable member facilitates at least one of the following: measurement, localization, selection and sampling of material from the transition zone of the intervertebral disc. It is contemplated that a wall of the implantable member may comprise at least one of a diffusion membrane, a frangible wall portion, and a wall portion having at least one perforation to permit placement of at least one treatment material from the implantable member into the transition zone of the intervertebral disc. For example, the implantable member may distribute at least one material from the implantable member to the transition zone thereby providing at least one localized infusion to nondestructively heal the intervertebral disc and/or to apply therapeutic material. In addition, in one embodiment the implantable member may distribute at least one material from the implantable member to the transition zone thereby providing at least one continuous localized infusion to nondestructively heal the intervertebral disc. It is further contemplated that the material of the implantable member comprises treatment material and the catheter is guidable.

Also disclosed herein is an implantation device comprising, in combination, an introducer member having an opening and a substantially flexible catheter located in an inner portion of the introducer member. The catheter may selectively extending from the inner portion of the introducer member through the opening of the introducer member. The catheter may be configured to both contain and selectively deliver an implantable member and the implantable member may be guided by the catheter along a path of an inner portion of a body receiving treatment. A portion of the implantation device may prevent retraction of the implantable member from the inner portion of the body when the catheter is retracted into the introducer member after implanting the implantable member.

In one configuration, an aspect of the body receiving treatment is selected from the group consisting of an intervertebral disc, a breast tissue, a skeletal bone, a portion of the alimentary canal, a portion of the brain and nervous system, a portion of the circulatory system and a portion of the respiratory system, or any hollow viscous, or potential space created from tissue or tumor removal. In one embodiment the path has a substantially circumferential shape and, the implantable member is substantially located in a substantially circumferentially shaped inner portion of the body. The implantable member may facilitate at least one of measurement, localization, selection and sampling of body organ tissue material from an inner portion of the body. It is further contemplated that a wall of the implantable member comprises at least one of a diffusion membrane, a self-dissolving membrane, a frangible wall portion and a wall portion having at least one perforation to permit placement of at least one treatment material from the implantable member into an inner portion of the body. The implantable member may comprise medication, tissue or stem cell transplantation or infusion, or both. It is contemplated that the catheter is inserted percutaneously into an inner portion of the body thereby causing minimal destruction of surrounding body tissue. It is further contemplated that at least a portion of the implantation device having at least one marker to permit visualization of the location of the catheter in the body by at least one technique selected from the group consisting of fluoroscopic x-ray, x-ray, computed tomography, magnetic resonance imaging and positron emission tomography. The implantable member may itself, secondary to its physical structure, provide stinting of an inner portion of the body receiving treatment to both support and medicate the inner portion of the body.

Also disclosed herein is a therapeutic material distribution method comprising the steps of guiding an implantable member catheter along a path of an inner portion of a body receiving treatment and implanting the implantable member into the inner portion of the body. Thereafter, preventing retraction of the implantable member from the inner portion of the body wherein a portion of the implantation material is left behind, while retracting the catheter into the introducer member after implanting.

In one variation, the method further comprises the steps of inserting the catheter into a predetermined portion of the body and advancing the catheter at a first rate into an inner portion of the body along the path. Thereafter, extruding tissue material from the path of the body thereby providing the path having a channel defined by the advancement of the catheter and retracting the catheter at a second programmable rate along the predetermined path. This allows for extruding the implantable member into the channel of the path while retracting the catheter.

In one embodiment this method further comprises implanting the implantable member in a substantially circumferential path or spiral path of an inner portion of the body to facilitate at least one of measurement, localization, selection and sampling of body organ tissue material from an inner portion of the body. Any portion of the body may be select including but not limited to providing an aspect of the body is selected from the group consisting of an intervertebral disc, a breast tissue, a skeletal bone, a portion of the alimentary canal, a portion of the brain and nervous system, a portion of the circulatory system, a portion of the respiratory system, cavity, canal, mass, and space occupying legion. The method may further comprise providing at least one marker which is used to visualize the location of the at least one marker in the body by at least one technique selected from the group consisting of fluoroscopic x-ray, x-ray, computed tomography, magnetic resonance imaging and positron emission tomography. Then extruding medication, tissue, stem cell material, or both from the catheter of the implantation device into a substantially circumferential path of an inner portion of the body.

Also disclosed herein is a device configured to apply electrical, magnetic, or chemical energy to a vertibre disc. Such an embodiment may comprise an introducer member having an opening and a substantially flexible catheter of the implantation device. The catheter is located in an inner portion of the introducer member and the catheter selectively extends from the inner portion of the introducer member through the opening of the introducer member. During use the catheter is configured to apply or insert into the disc an apparatus that may apply electrical, magnetic, or chemical energy to the disc, wherein apply electrical energy, magnetic energy, or chemical substances to the disc selectively stimulates or promotes intradisc function or delivery of material into our out of the disc.

In one embodiment the chemicals comprise antibiotics. In one embodiment the device is configured to pump material into or out of the disc in connection with disc respiration.

The foregoing and other articles, features, and advantages of the invention will be apparent from the following more detailed description of the preferred embodiments of the invention, as illustrated in the accompanying drawings. The various features may be utilized or claimed alone or in any combination.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 is a perspective view showing a vertebral column of a person with three designated principal regions of the vertebral column.

FIG. 1A is an exploded perspective view showing a more detailed view of a neck vertebra from an upper region surrounding a spinal column of the vertebral column of FIG. 1.

FIG. 1B is an exploded perspective view showing a more detailed view of a thoracic vertebra from a mid-region surrounding the spinal column of the vertebral column of FIG. 1.

FIG. 1C is an exploded perspective view showing a more detailed view of a lumbar vertebra from a lower region surrounding the spinal column of the vertebral column of FIG. 1.

FIG. 2A is a sectional view showing a cross section of an intervertebral disc of the vertebral column of FIG. 1.

FIG. 2B is a sectional view showing a cross section of a herniated intervertebral disc of the vertebral column of FIG. 1.

FIG. 2C is a sectional view through a portion of the vertebral column of FIG. 1 depicting how the herniated intervertebral disc of FIG. 2B touches a nerve proximate to a spinal chord of the vertebral column of FIG. 1.

FIG. 3A is an exploded perspective view showing the intervertebral disc of FIG. 2A coupled to a pair of vertebrae of the vertebral column of FIG. 1 with a shaft of a needle inserted into the intervertebral disc.

FIG. 3B is an exploded sectional view along viewing direction 3B-3B showing the shaft of the needle of FIG. 3A inserted within a nucleus pulposus of the intervertebral disc of FIG. 3A.

FIG. 4A is a sectional view depicting a nucleus pulposus, an annulus pulposus and a transition zone of an intervertebral disc.

FIG. 4B is an a perspective cross sectional view of the intervertebral disc of FIG. 4A including an introducer member of an intervertebral disc implantation device having an opening and a catheter disposed therein positioned to deliver an implantable member into the transition zone of the intervertebral disc of FIG. 4A.

FIG. 5A is an elevational view depicting a portion of an intervertebral disc implantation device and the catheter within the opening of the introducer member of FIG. 4B.

FIG. 5B is an elevational view depicting a portion of the intervertebral disc implantation device and the catheter of FIG. 4B with the catheter and a portion of an implantable member extending circumferentially from the catheter for implanting into a patient.

FIG. 5C is an elevational view depicting a portion of the intervertebral disc implantation device and the catheter of FIG. 4B with the implantable member dispensed from the catheter and with the catheter in the opening of the introducer member after the implantable member is implanted.

FIG. 6 is a sectional view depicting a breast of a person with the catheter of an implantation device circumferentially disposed within a portion of the breast in preparation for implantation of an implantable member.

FIG. 7 is a sectional view depicting a body organ of a person with the catheter of the implantation device of FIG. 6 circumferentially disposed within a portion of the body in preparation for implantation of an implantable member.

FIG. 8 is a perspective view depicting a body portion (such as a portion of a bowel or a duct) of a person with an implantable member circumferentially disposed within a portion of the body.

FIG. 9 is an exploded perspective view depicting a bone of a person with the catheter of the implantation device of FIG. 6 disposed around another implant within a portion of the broken bone to deliver an implantable member to a specific portion of the bone.

### DESCRIPTION OF THE INVENTION

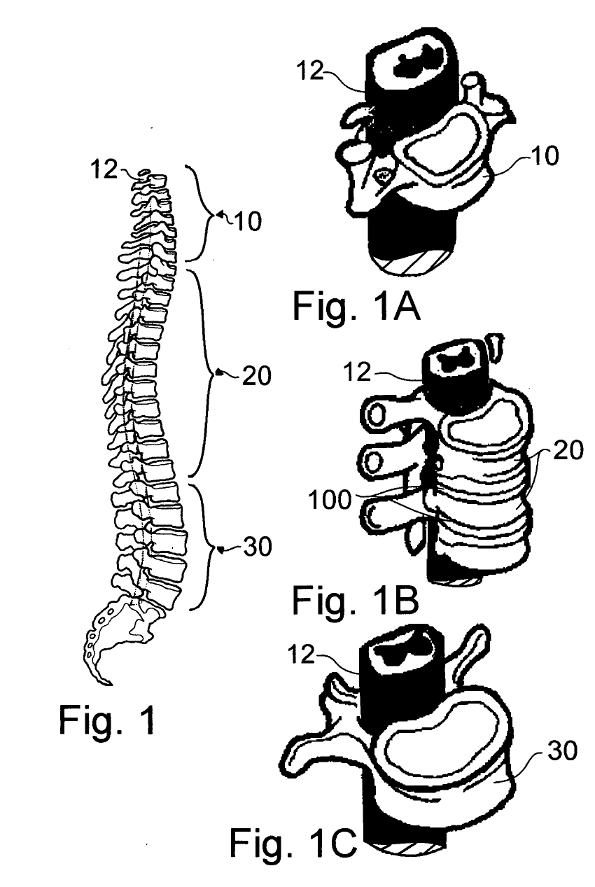

According to FIG. 1, the spine of a person comprises a vertebral column having a plurality of vertebrae in three principal vertebral regions. The principal vertebral regions are a plurality of neck vertebrae 10, a plurality of thoracic vertebrae 20, and a plurality of lumbar vertebrae 30 with a spinal chord 12 running substantially from the upper portion of the spine (neck) to the lower portion of the spine (lumbar) located in a protected region of each one of the principal vertebral regions. FIG. 1A is an exploded perspective view showing a more detailed view of a neck vertebra 10 of the plurality of neck vertebrae 10 (shown in FIG. 1) surrounding the spinal chord 12 of the vertebral column. The spinal chord 12 is located in an opening of a protected region of the neck vertebra 10. FIG. 1B is an exploded perspective view showing a more detailed view of a plurality of thoracic vertebrae 20 (also shown in FIG. 1) surrounding the spinal chord 12 of the vertebral column. The spinal chord 12 is located in an opening of a protected region of the thoracic vertebrae 20. An intervertebral disc 100 is located between each one of the thoracic vertebrae 20. It is understood that an intervertebral disc 100 is located between each one of the vertebrae of the vertebral column. The intervertebral discs 100 absorb stress and shock incurred in a body of a person during movement and prevents each of the plurality of vertebrae of the vertebral column from grinding against one another. The intervertebral discs 100 may serves as shock absorbers, giving resilience to the spinal column as well as flexibility to the vertebral column. The disc may include a transition zone and end plate zones. Growth may occur in the transition zone and the end plate zones, which may contact the vertibre may allow for material transfer, such as but not limited water, nutrients, or hormones, into or out of the disc. It is contemplated that the disc may communicate with other aspects of the body. By way of example, pressure or hormonal transfer may occur between the disc and the body to facilitate disc growth or malfunction. A respiration or pumping of the disc may occur across cycles to facilitate the respiration.

When the person is erect, the various portions of the intervertebral disc 100 are under uniform pressure, but when the spine is flexed, extended or bent to a side, a portion of each intervertebral disc 100 experiences increased compression whereas another portion of each intervertebral disc 100 experiences increased tension. The inner portion of each intervertebral disc 100 may also experience shear forces.

FIG. 1C is an exploded perspective view showing a more detailed view of a lumbar vertebra 30 of a plurality of lumbar vertebrae 30 (shown in FIG. 1) surrounding the spinal chord 12 of the vertebral column. Most lower-back injuries occur in the region of the plurality of lumbar vertebrae 30 and more particularly in at least one intervertebral disc 100 located between a pair of lumbar vertebrae 30.

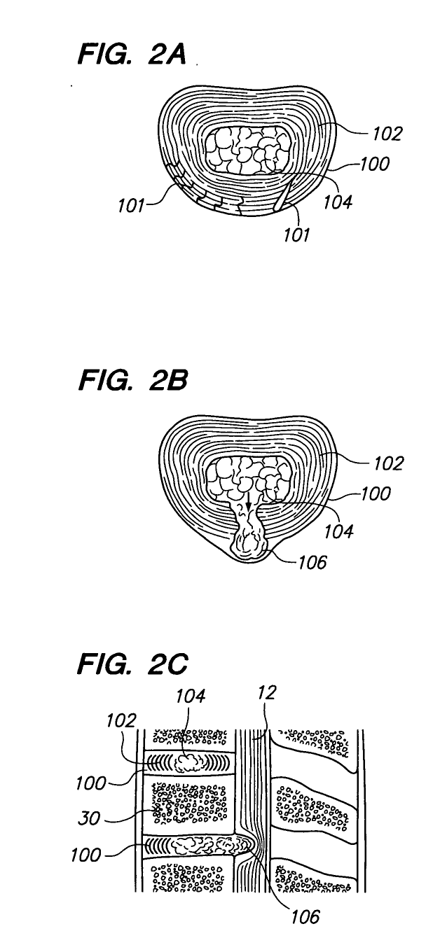

FIG. 2A is a sectional view showing a cross section of an intervertebral disc 100 of the vertebral column (see FIG. 1). An interior portion of the intervertebral disc 100 comprises an annulus fibrosus 102 and a nucleus fibrosus 104. The nucleus pulposis is the ‘hydrolic fluid’ that distributes vertical stress or pressure outward in all directions into the annulus and end plates. The annulus fibrosus ‘contains’ or surrounds the nucleus material. The annulus fibrosus 102 is a layered structure comprising water and sturdy elastic collagen fibers. The collagen fibers are oriented at different angles with similar construction to that of a radial tire. Collagen comprises fibrous bundles made of protein bound together by a gel comprising proteoglycans. Proteoglycans are bioactive long chain polymers comprising glycosaminoglycans (GAGs) covalently coupled to core proteins found in all connective tissues, extracellular matrix (ECM) and on the surfaces of many cell types. It is contemplated that the cells (chondrcytes) of the transition zone may be producing these materials. Glycosaminoglycans are long unbranched molecules containing a repeating disaccharide (sugar) unit. Usually one sugar is an uronic acid (either D-glucuronic or L-iduronic) and the other is either GlcNAc or GalNAc. One or both sugars comprise sulfate groups (the only exception being hyaluronic acid). GAGs are highly negatively charged which is believed to be essential for their function. Specific GAGs OF physiological significance include: Hyaluronic acid (D-glucuronate+GlcNAc), which occurs in synovial fluid, ECM of loose connective tissue; Dermatan sulfate (L-iduronate+GlcNAc sulfate), which occurs in skin, blood vessels and heart valves; Chondroitin sulfate (D-glucuronate+GalNAc sulfate), which is the most abundant GAG and occurs in cartilage, bone and heart valves; Heparin and Heparin sulfate (D-glucuronate sulfate+N-sulfo-D-glucosamine), which occurs as a component of intracellular granules of mast cells lining the arteries of the lungs, liver and skin in the case of Heparin and as basement membranes and as a component of cell surfaces in the case of Heparin sulfate; and Keratan sulfate ( Gal+GlcNAc sulfate), which occurs in cornea, bone and cartilage and is most often associated with Chondroitin sulfate.

Returning to FIG. 2A, the intervertebral disc 100 is the largest structure in the body of a person without a vascular supply. It is thought that intervertebral discs 100 absorb needed nutrients by osmosis or diffusion, or other mechanism. It is contemplated that the end plates may regulate the flow of material, such as nutrients, hormones, etc, into the disc. It is believed that the nucleus pulposus 104 comprises various ingredients including water, collagen and proteoglycans. It is believed that the nucleus pulposus 104 has different concentrations of each one of these ingredients compared to the annulus fibrosis 102. It is believed that the nucleus pulposus 104 comprises a greater quantity of water than the annulus fibrosis 102 and that the chemical make up of the nucleus pulposus is not compatible with surrounding tissue if released.

The disc may degenerate or be injured thereby causing small or large defect in the disc 100. The defect 101 may affect nerves located in the outer regions of the disc. In many instances, these types of micro defects may be the primary source of pain. In contrast to major disc repair, using the methods and apparatus described herein, these types of defects may be healed or regenerated.

FIG. 2B illustrates a sectional view showing a cross section of a herniated intervertebral disc of the vertebral column of FIG. 1. According to FIG. 2B a portion of the nucleus pulposus 104 may erupt through a portion of the annulus fibrosis 104 to form an outgrowth (hernia) portion 106 of the intervertebral disc 100. While in many cases such an eruption (hernia) is treated by appropriate rest and the use of anti-inflammatory medications applied either topically or taken orally, nevertheless a percentage of such eruptions require more aggressive treatment. In other instances, other undesirable issues may arise with regard to the intervertebral disc 100 that may require treatment.

FIG. 2C illustrates how the eruption of an intervertebral disc 100 causes pain to a person. According to FIG. 2C, when the intervertebral disc 100 (see FIG. 2B) erupts, the outgrowth (hernia) portion 106 of the intervertebral disc 100 contacts a nerve proximate to the spinal chord 12 or a portion of the spinal column 12, thereby causing pain to the person. This may be referred to as a ruptured or herniated disc.

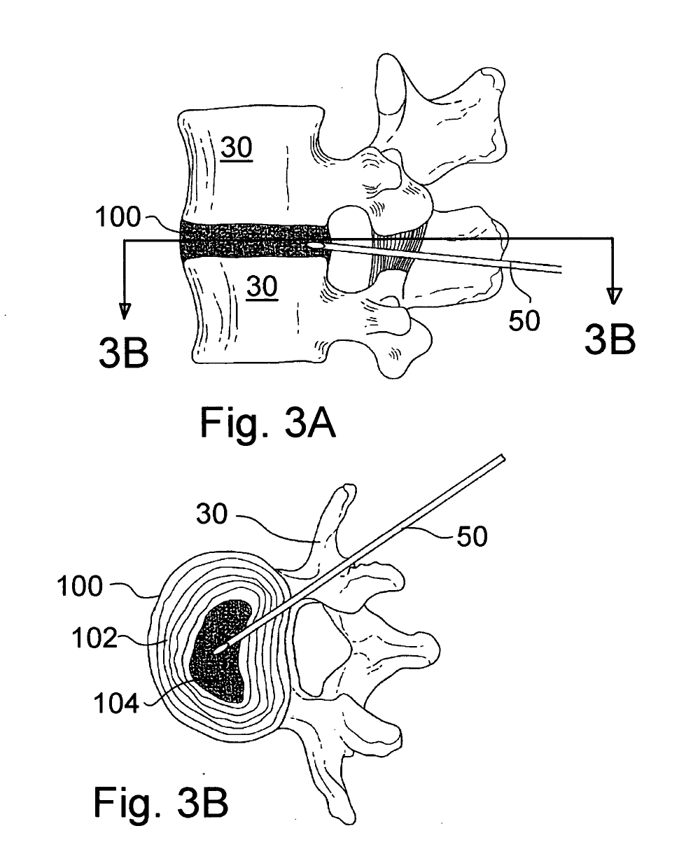

FIG. 3A is an exploded perspective view showing the intervertebral disc 100 (see FIG. 2A) coupled to a pair of vertebrae of the vertebral column (see FIG. 1) with a shaft of a needle 50 inserted into the intervertebral disc 100. In treatments involving percutaneous injection of medication to an interior portion of an intervertebral disc 100, it is common to inject the needle 50 into the nucleus pulposus 104 of the intervertebral disc 100.

FIG. 3B is a sectional view along the line 3B-3B of FIG. 3A illustrating that the point of the needle 50 passes through the wall of the intervertebral disc 100 and thereafter through the annulus fibrosis 102 and into the nucleus pulposus 104. In addition to lysis (chemical dissolving) other treatment techniques include mechanical removal, electro-thermal, radio frequency electric currents, laser and ultrasound via penetration of the intervertebral disc 100 with a needle 50 to treat the intervertebral disc 100 when it is herniated by relieving pressure within the intervertebral disc 100. Such treatments suffer from the disadvantage of substantially destroy the living cells of the inner portion of the intervertebral disc 100. As can be appreciated, the method and apparatus disclosed herein is superior to destructive techniques of the prior art.

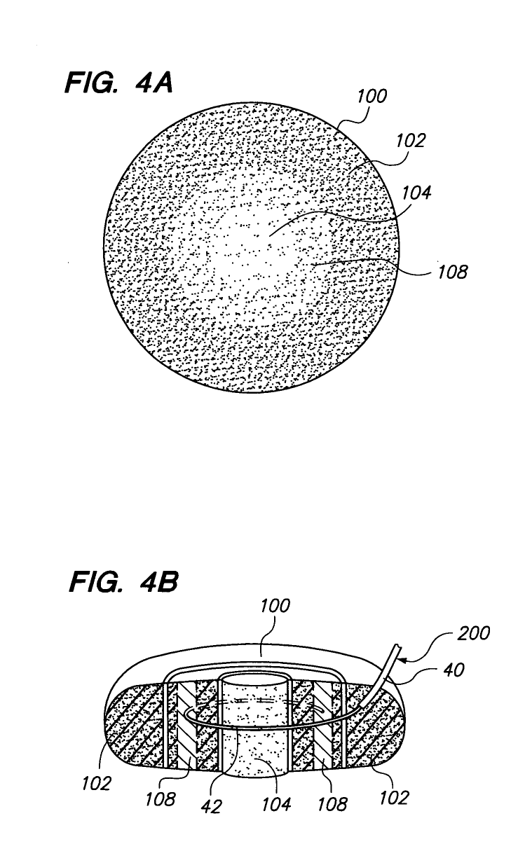

According to FIG. 4A an interior portion of the intervertebral disc 100 is a much more complex structure than shown in FIG. 2A (see description above) and more than is contemplated by prior art treatment methods. Apart from the previously described annulus fibrosis 102 and the nucleus pulposus 104, a region located substantially between the annulus fibrosis 102 and the nucleus pulposus 104 comprises a transition zone 108. It is believed that the cells comprising the transition zone 108 have different morphology and viscoelastic properties compared to those of the annulus fibrosis 102 and the nucleus pulposus 104. For example, the molecular weight of the proteoglycans is believed to be different in each one of the annulus fibrosis 102, the transition zone 108, and the nucleus pulposus 104. Changes in the morphology (and the chemistry) of the transition zone 108 may explain some of the degradations that occur within the intervertebral disc 100. Therefore, treatments that are specific to the transition zone 108 may be substantially more effective to treat the intervertebral disc 100 thereby avoiding the current procedures which substantially destroy the inner portion of the intervertebral disc 100 to relieve pain. It is contemplated that the intervertebral disc 100 may be treated to restore growth and activate a rebuilding re-growth process, reduce symptoms, repair damage, restore homeostasis, and/or simulate regrowth. Thus, in contrast to destroying the intervertebral disc 100, treatment may be made to repair or heal one or more portions of the disc structure.

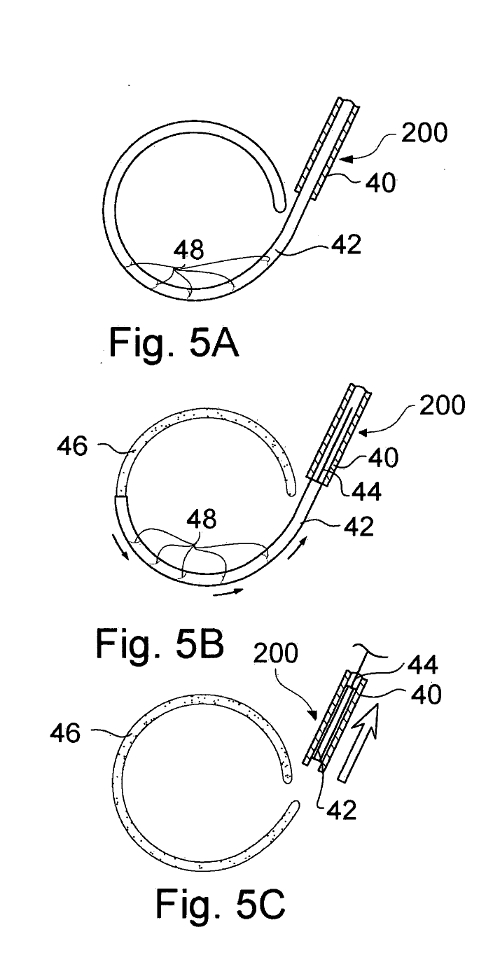

Referring now to FIG. 4B, an intervertebral disc implantation device 200 comprises an introducer member 40 having an opening. FIGS. 5A-5C may also be consulted to aid in understanding of this example embodiment. In one embodiment the introducer member 40 comprises a stiff or flexible shaft has a hollow center area and is capable of being inserted into a body. The distal end (which is inserted into the body) of the introducer member 40 has an opening through which material or additional apparatus may extend and/or retract. In one embodiment the introducer member 40 further comprises a catheter 42. It is contemplated that in one embodiment the catheter 42 of the introducer member 40 may have a preformed shape. The catheter 42 may comprise or be constructed of plastic, a composite material, a metal and the like, or any other material or a combination of any of these materials. For example, a suitable metal may comprise spring metal or superelastic memory material (such as a nickel-titanium alloy, i.e. Nitinal™.

The catheter 42 and introducer member 40 may comprise a rigid or substantially flexible material and the material comprising the catheter is characterized by having sufficient flexibility to follow an inner portion of the annulus fibrosis 102 proximate to the transition zone 108. Preferably an end of the catheter 42 located in the transition zone 108 does not penetrate into the annulus fibrosis 102 after the introducer member 40 first penetrates the annulus fibrosis. In one embodiment the introducer member 40, the catheter 42, or both may be steerable. Steerable catheters are generally known and hence not described in detail.

The catheter 42 may be located in an inner portion of the introducer member 40 and the catheter may be selectively extended from the inner portion of the introducer member through the opening in the distal end of the introducer member.

In one embodiment an implantable member 46 (see FIGS. 5B and 5C, and the description below) may be contained within and guided by the catheter 42 along a path of an inner portion of the intervertebral disc 100. A machine or person may guide the implantable member 46, the catheter 42, or both into the inner portion of the intervertebral disc 100.

In one embodiment it is contemplated that the path is predetermined. Furthermore, the path may have a substantially circumferential shape. In one embodiment, the path may be entirely or partially contained within or near the transition zone 108 which lies generally between the 104 nucleus pulposus and the annulus fibrosis 102 of the intervertebral disc 100. It is contemplated that the transition zone may comprise a zone within the disc 100 of particular importance because it may be capable of re-growth or regeneration, particularly in the presence of treatment material. The transition zone includes living cells of the disc, which may re-supply or regenerate new material for the nucleus pulposis and the annulus. These chondrocites of the transition zone may maintain the disc.

The term treatment material may comprise, and is not limited to, any material listed herein, replacement materials, nutrition or nutritive solutions, regenerative solutions, continuous release or time release solutions or gels, hormones or other materials that stimulate growth of existing cells, or growth hormones, tissue, stem cells or other type cells medication, chemicals, radiation source, sterilizer, infection treatment, pre-treatment preparation prior to stem cell or other treatment option, antibiotics such as for discitis, treatment. It is contemplated that stem cells are defined as cells derived from any location or source. The term stem cell, cell infusion, and cell may be used interchangeably herein.

The treatment material may also comprise regenerative solutions, gels, slurries, or solids. By way of example and not limitation, the regenerative solution may comprise one or more chemicals, such as but not limited to DMSO. The regenerative solution may comprise biochemical growth factors, cartilage factors, enzymes, hormones, proteins. These may be isolated and injected or infused in to the disc along, in combination, or a combined soup type mixture. The regenerative solution may comprise nutritional factors, such as building blocks for growth, or stimulation factors to encourage or force existing cell to produce or grow. The regenerative solution may comprise stem cells or material to prepare the disc to accept stem cells.

A portion 44 (typically a blocker, a plunger or a stylet and the like as shown in FIGS. 5B, 5C and described below) of the intervertebral disc treatment device 200 prevents or hinders retraction of the implantable member 46 from the inner portion of the intervertebral disc 100 when the catheter 42 is retracted into the introducer member 40 after implanting the implantable member (see FIG. 5C and the description below). The implantable member 46 may be substantially located in a substantially circumferentially shaped portion of the transition zone 108 of the intervertebral disc 100. The implantable member 46 may facilitate at least one of measurement, localization, selection and sampling of material from the transition zone 108 of the intervertebral disc 100 by acting as a probe, sampler or as a delivery device for treatment material.

When the implantable member 46 is a delivery device, a wall of the implantable member may comprise at least one of a diffusion membrane, a frangible wall portion and a wall portion having at least one perforation to permit placement of at least one material from the implantable member into the transition zone 108 of the intervertebral disc 100. The implantable member 46 may seep or slowly diffuse and distribute at least one material from the implantable member to the transition zone 108 thereby providing at least one localized infusion to nondestructively heal the intervertebral disc 100. The material of the implantable member 46 may comprise and is not limited to at least one of the following: at least one medication and at least one stem cell infusion implanted in the transition zone 108 of the intervertebral disc 100. It is understood that a combination of both medication and stem cell infusion may be implanted. A suitable medication may include antibiotics, anti-inflammatory drugs, growth hormones and the like. These materials may be referred to as treatment material. The implant member 46 may be encapsulated in a casing which dissolves over time or may comprise a material which may provide structural enhancements to the interior of the intervertebral disc 100 (such as stinting). The implantable member 46 comprises at least one material selected from the group consisting of a fluid, a slurry, a semi-solid, a gel, a matrix and a solid. In one embodiment, such as in the case of a fluid or slurry, the treatment material may be injected while the catheter 42 is inserted or removed from the treatment area.

It is contemplated that the implant into the disc may comprise an electrical, chemical, chelation agent or magnetic type implant selected to treat the disc, stimulate growth/repair as described herein. These elements may also be used to draw or drive material into or out of the disc. Also insertable into the disc is a single point or multi-point pressure monitor capable of monitoring pressure or pressure gradients within the disc.

It is also contemplated that the disc may naturally execute a pump operation or perform ‘respiration’, such as loading/unloading or expansion/contraction, during cycles, such as daily or other time frame. During this respiration cycles, water, chemicals, oxygen may be introduced into the disc. Using the method and apparatus described herein, treatment material may be introduced in connection with these natural cycles to improve or achieve treatment. By way of analogy, but not limitation, medication introduced into the lungs is best introduced during a breath intake. Likewise, the method and apparatus described herein may introduce treatment material during either of the loading/unloading or compression/expansion from the disc.

FIG. 5A is an elevational view depicting a portion of the intervertebral disc implantation device 200 and the catheter 42 within the opening of the introducer member 40 (see FIG. 4B and description above). It is understood that in another embodiment of the method and apparatus disclosed herein an implantation device 200 has the same features as the intervertebral disc implantation device 200 (as described above), except that the catheter 42 of the implantation device 200 may have a different shape, functionality and length according to the need to place the catheter in a different portion of a body of a person. In the embodiment shown in FIGS. 5A, B and C, the implantation devices 200 may be used for disc treatment or, when configured in other shapes or sizes, configured to treat other areas of a body. At least a portion of the intervertebral disc implantation device 200 (or the implantation device 200) has at least one marker 48 to permit visualization of the location of the catheter 42 in the intervertebral disc 100 by at least one technique selected from, but not limited to the group comprising fluoroscopic x-ray, x-ray, computed tomography, magnetic resonance imaging and positron emission tomography. It is understood that the marker 48 may be located on a portion of the catheter 42 or on another portion of the intervertebral disc implantation device 200 to indicate relative position of the catheter 42 within the intervertebral disc 100. Preferably, the marker 48 is located on a portion of the catheter 42 to permit accurate placement of the catheter 42 into an inner portion of the intervertebral disc 100. It is contemplated that the term “intervertebral disc” may be interchangeable with the term “body organ” or body (see description below) since similar concepts apply in multiple embodiments of this disclosure. It is further understood that the marker 48 may comprise a plurality of markers 48 having graduations of a predetermined size. The marker 48 may comprise a material that facilitates fluoroscopic observation thereby enhancing accurate placement of the catheter 42. The marker 42 may also be used to selectively alter the amount of the implantable member 46 to be implanted into an inner portion of the intervertebral disc 100.

Turning to FIG. 5B, a view is shown wherein the implantable member 46, which may comprise any treatment material, is left behind during retraction of the catheter 42 into the introducer member 40. A blocker portion 44 comprising, but not limited to, at least one of the following: a blocker, a plunger, a stylet and the like of the intervertebral disc implantation device 200 (or the implantation device 200) prevents retraction of the implantable member 46 from the inner portion of the intervertebral disc 100, or other body part, when the catheter 42 is retracted into the introducer member 40 after implanting the implantable member. The portion 44 also may facilitate extrusion of the implantable member 46 along the predetermined path into the inner portion of the intervertebral disc 100.

As described above, in one embodiment the path has a substantially circumferential shape and the implantable member 46 is located in a substantially circumferentially shaped portion of the transition zone 108 of the intervertebral disc 100. The implantable member 46 may also be in a spiral shape or any other shape to achieve treatment. It is understood that the implantable member 46 may also have a substantially circumferential shape when implanted in the transition zone 108. Since the implantable member 46 is localized after implantation in the transition zone 108, treatment of the intervertebral disc 100, or other body area, with at least one of medication and stem cell infusion, or both or any other treatment material to provide enhanced healing.

FIG. 5C depicts a portion of the intervertebral disc implantation device 200 (or the implantation device 200) with the catheter 42 fully retracted through the opening of the intervertebral disc implantation device 200 and the portion 44 (the blocker, the plunger, the stylet and the like) and with the implantable member 46 fully dispensed from the catheter after implantation. At this stage and configuration, the person or machine may remove the introducer member 40, including the catheter 42 from the body.

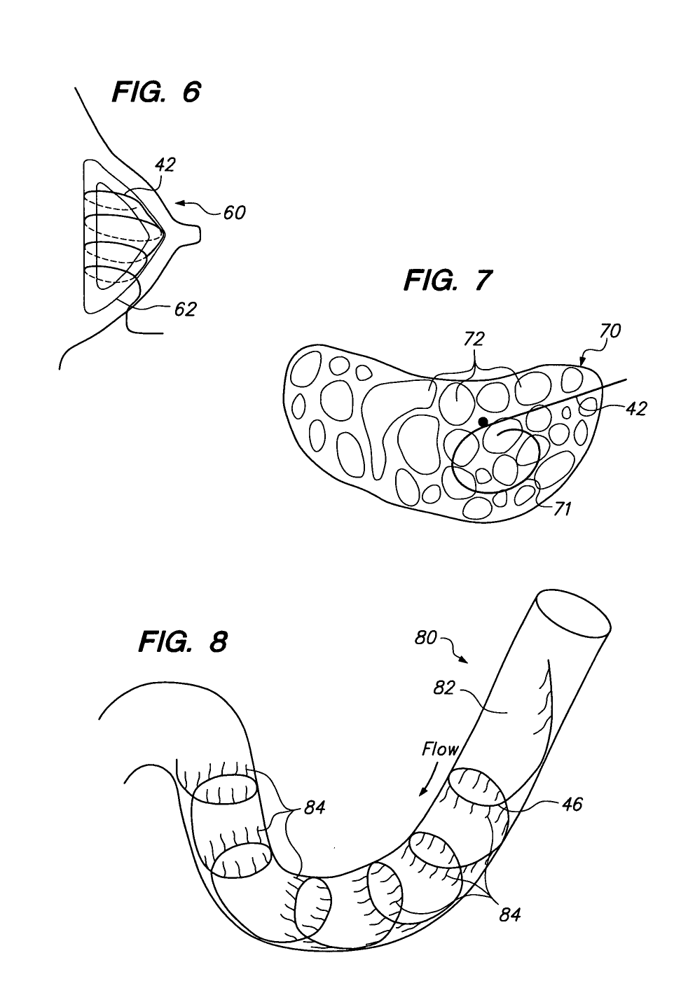

Turning now to FIGS. 6, 7, and 8 numerous other embodiments of the implantation device are shown. In these examples, the catheter 42 is configured to both contain and selectively deliver an implantable member 46. The implantable member 46 is guided by the catheter 42 along a path of an inner portion of a body receiving treatment. As described above, a portion 44 of the implantation device 200 prevents retraction of the implantable member 46 from the inner portion of the body when the catheter 42 is retracted into the introducer member 40 through the opening of the introducer member after implanting the implantable member. The portion 44 of the implantable device 200 comprises but is not limited to at least one of the following: a blocker, a plunger and a stylet.

It is contemplated that any body part may receive treatment, including but not limited to an intervertebral disc 100, a breast tissue 60, a skeletal bone 90, a portion of the alimentary canal 80, a portion of the brain, nervous system, or respiratory system 70, a portion of the circulatory system (FIG. 8 depicts either a portion of the alimentary system or a portion of the circulatory system comprising veins and arteries or any other similar structure) and a portion of the brain, nervous system, or respiratory system 70 (FIG. 7 depicts either a portion of the brain and nervous system or a portion of the respiratory system, or any other organ).

It is contemplated that the implantable member 46 may be placed in a substantially circumferential path or in any path other than a single point localized injection site to enhance healing of the body organ by providing greater distribution and be more therapeutically effective to a specific portion of the body organ.

Referring to FIG. 6, the inner portion of the body comprises the breast tissue 60 having a cavity or tumor 62. The catheter 42 of the implantation device 200 is circumferentially disposed about the cavity or tumor 62 of the breast tissue 60 in preparation for implantation of the implantable member 46. Since the implantable member 46 surrounds the cavity or tumor 62, opportunity exists for greater distribution and more localized therapeutically effective treatment of the region surrounding the cavity or tumor along the path defined by advancement of the catheter 42 around the cavity or tumor.

In previous treatment methods, medication would tend to accumulate in a lumen portion of the body resulting in ineffective treatment of the cavity or tumor 62 of the breast tissue 60 or the surrounding breast tissue. As shown in FIG. 6, the path has a substantially circumferential shape and the implantable member 46 is substantially located in a substantially circumferentially shaped inner portion of the body (the breast tissue 60). It is understood that when viewed as a three dimensional structure, the path may be substantially spiral or other shape to achieve optimal treatment. The catheter 42 may comprise a material (see description above) having a predetermined memory to specifically conform to an outer portion of the cavity or tumor 62, or may be preformed to assume a shape similar to the cavity or tumor. The implantable member 46 (not shown in FIG. 6, but similar to the implantable member 46 of FIGS. 5B and 5C) facilitates at least one of measurement, localization, selection and sampling of body organ tissue material from an inner portion of the breast tissue 60 of the body.

It is contemplated that where it is desirable to remove tissue because of a suspicion of malignant cell growth, the implantable member 46 may be adapted to both remove a portion of suspected malignant cells, permit immediate analysis of suspected malignant cells, and also provide a path via the advancement of the catheter 42 for insertion of medication, stem cell infusion, or both.

It is also contemplated that in the various embodiments shown and described herein, the treatment material may be provide on the inside surface of a cavity, such as for example a hollowed out tumor. In such an embodiment the implantable member 46 comprising at least treatment material may be placed along the inner surface of the cavity 62. In one embodiment the implantable member 46 is configured to expand outward to contact the surface of the cavity 62. This is shown generally in FIG. 6 in that the implantable member 46 may tend to expand outwardly thereby contacting the inner surface of the cavity 62. This provide a treatment option, thereby dispersing the treatment material to the inner surface of the cavity 62. As can be appreciated this has numerous advantages over a local spot injection of treatment material.

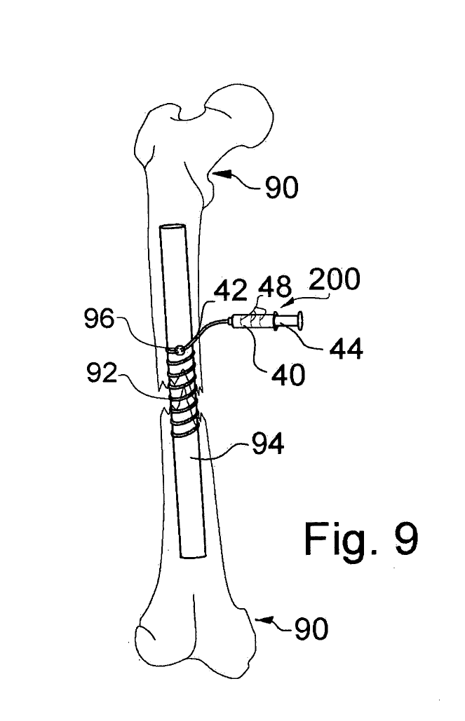

FIG. 7 illustrates a sectional view depicting a body organ 70 of a person with the catheter 42 of the implantation device circumferentially disposed within a portion of the body organ 70 in preparation for implantation of an implantable member 46 (as described in FIGS. 4B, 5A, 5B, 5C and 6 above and in FIGS. 8 and 9 below). It is understood that the body organ 70 of FIG. 7 may be any suitable body organ as described above where it is desirable to implant the implantable member 46 comprising any treatment material. The insert 71 may be configured to flex with the lung or organ. The insert 71, which may comprise an implantable member or treatment material, may spring outward thereby maintaining in an open or un-collapsed state the organ or cavity. The insert 71 may be steerable to be formed into a curved or circular shape. The medication may be released immediately or as a slow release material designed to be uniformly distributed in an inner portion of the body organ 70 and to provide therapeutic healing of a localized or expanded region of the body organ. Typically the medication, tissue or stem cell infusion or transplant, or both is absorbed by the localized region of the inner portion of the body organ 70 receiving the treatment. It is contemplated that the medication, tissue or stem cell infusion or transplant, or both may be further distributed to other portions of the body depending on the nature of the body organ 70.

The path of the catheter 42, implantable member 71, and treatment material 71 may comprise any shape or path to achieve optimal treatment. In one embodiment the path has a substantially circumferential shape with the implantable member 46 substantially being located in a substantially circumferentially shaped inner portion of the body or body organ 70. However, it is understood that other suitable path geometries may provide additional benefits. The most preferred path geometry achieved by the advancement of the catheter 42 in an inner portion of the body organ 70 is one which provides that the localized surface area of the implantable member 46 most effectively treats the body organ 70 for the specific treatment required for the body organ. It is understood that this treatment varies according to the properties of the body organ and the properties of the implantation device 200.

In one embodiment the organ of FIG. 7 comprises a lung and as such, it may be desired to implant a flexible implantable member 71 that while providing treatment material also expands or outwardly flexes to assume the shape of the lung. The implantable member may therefore provide such a flexible structure that may expand outwardly to contact the inner surface of the lung to provide treatment material to the lung over a larger area than a single spot injection. Moreover, implanting a implantable member may be less traumatizing to the patient than numerous spot injections of treatment material. In addition, a catheter and treatment method as described herein may prevent or make less likely a blockage of the catheter, such as may be associated with a catheter having a single outlet. Likewise, it is less likely scar tissue or other growth tissue would block the multiple outlet catheter.

In one embodiment, the catheter 42 is inserted percutaneously into an inner portion of the body thereby causing minimal destruction of surrounding body tissue of the body organ 70. As shown in FIG. 7 the body organ 70 may comprise a plurality of substructures 72, with each substructure of the body organ 70 having a different functionality. According to the above description, the introducer member 40 would permit selective advancement of the catheter 42 along a path to surround the appropriate targeted substructure 72 without destroying surrounding tissue. The catheter 42 may be steerable or selectively directed by a person or by a machine. In one embodiment the catheter 42 may be configure to also facilitate one or more of the following: measurement, localization, selection and sampling of tissue material from the region surrounding the body organ 70.

Additionally, after analysis, a user of the catheter 42 may implant the implantable member 46 along a portion of the path surrounding the targeted inner portion substructure 72 of the body organ 70 to distribute at least one material from the implantable member to the inner portion substructure of the body organ. This may be done for any reason including to either provide at least one localized infusion to nondestructively heal the body organ 70 of the body or to provide a continuous localized infusion to nondestructively heal the body organ of the body. Hence, in any embodiment disclosed herein, the treatment material may diffuse at any rate to provide a selective and controllable rate of treatment. For example, in one embodiment in order to distribute the medication or stem cell infusion (as described above), a wall of the implantable member 46 may comprise at least one of a diffusion membrane, a self-dissolving membrane, a frangible wall portion and a wall portion having at least one perforation to permit placement of at least one material from the implantable member into an inner portion of the body organ 70 of the body. It is contemplated that implantable member 46 may comprise at least one material selected from the group consisting of a fluid, a slurry, a semi-solid, a gel, a matrix and a solid. It is understood that the fluid may comprise a liquid mixture, an aerosol or a gas. The implantable member 46 may serve as a conduit for placement and delivery by distribution of a material comprising medication, stem cell infusion, growth hormone and the like, or a combination of any of the material. It is understood that more than one implantable member 46 may be implanted by the catheter 42 to provide greater coverage or treatment.

FIG. 8 depicts a body organ 80 (such as a portion of a bowel or a duct) of a person with the implantable member 46 circumferentially disposed within a portion of the body organ. It is also contemplated that the implantable member 46 may provide stinting as well as providing an implantable material to a wall of the body organ 80. The body organ 80 may comprise a portion of the alimentary canal of a body of a person or a portion of the circulatory system such as a vein or an artery of the body of the person. As shown in FIG. 8 the implantable member is located in an inner portion 82 of the body organ 80. The inner portion 82 is generally referred to as the lumen (or cavity) of the body organ 80 and is a conduit for flow of material (such as chyme in the case of the alimentary canal and of blood constituents in the case of the circulatory system) along the inner portion of the body organ.

As a benefit to the method and apparatus described herein, the flow of material in the body organ 80 as shown in FIG. 8 results in an advanced and complete distribution of medication, stem cell infusion, or both into various regions of the body of the person. Referring to FIG. 8, the implantable member 46 distributes material 84 from the implantable member into the flow path of the inner portion of the body organ 80. Because the wall of the implantable member 46 has multiple sites for distribution of medication, stem cell infusion, or both, into the flow path of the inner portion of the body organ 80 there is less opportunity for accumulation of medication, stem cell infusion, or both in a lower portion of the lumen, in contrast to current techniques. More complex flow patterns may exude from the implantable member 46 depending on the release rate of material from the wall of the implantable member.

Additionally, the stinting feature of the implantable member 46 may provide greater opening of the inner portion 82 of the body organ 80 to support the body organ or to open up an occlusion in the inner portion of the body organ. The implantable member 46 may comprise a material that more effectively removes the occlusion in the inner portion 82 of the body organ 80. It is contemplated that the implantable member 46 may also comprise material that is more bio-compatible.

In all embodiments shown and described herein, it is contemplated that the implantable member 46 may comprise the treatment material itself or structure configured to disperse the treatment material. For example, in one embodiment the implantable member 46 may comprise a hollow flexible tube having an array of holes therein. A syringe or other device may be utilized to selectively push material through the hollow tube and out through the array of small holes in the implantable member 46. This provides a diffused injection method over a wide treatment area. The implantable member 46 may be removed after each treatment or remain inside the body for repeat treatment over time.

FIG. 9 is an exploded perspective view depicting a broken bone 90 of a body of a person with the catheter 42 of the implantation device circumferentially disposed around a pin 94 or any other structure (another implant) within an inner portion of the broken bone. In this configuration the catheter 42 may deliver an implantable member 46 and/or other treatment material(not shown in FIG. 9, but shown in FIGS. 5B and 5C, and described above) to a specific portion of the broken bone 90.

In practice of one embodiment, the pin 94 is placed along a path defined by a substantially longitudinal axis of an inner portion of the broken bone 90. It is understood that other paths may be more preferable when the broken bone 90 has a different fracture profile. In current treatments, the pin 94 may have any type medication, such as but not limited to, growth hormone or antibiotic on a portion of an outer surface of the pin 94. Such current treatments do not result in highly specific and directed medication of the broken bone 90. According to the current disclosure, in one embodiment, a tube 92 may be disposed about the pin 94 along a substantially circumferential path surrounding the pin. In other embodiments other paths may be selected. An end of the tube 92 which is not inside the body may be configured with an opening configured with a coupler 96 adapted to couple to an end of the catheter 42 of the implantation device 200 and may also couple to syringe as shown The combination of the implant 94 and the tube 92 are implanted into an inner portion of the broken bone 90.

Subsequently, the end of the catheter 42 is coupled to the coupler 96 of the tube 92. Medication, stem cell infusion, or both are transferred from the catheter 42 to the tube 92 which may comprise a bio-active material that disintegrates to release material such as medication, stem cell infusion or any treatment material. It is understood that the tube 92 may have a second opening to permit sampling of tissue from an inner portion of the broken bone surrounding the pin 94 by passing a portion of the catheter 42 through the tube 92. Furthermore, the catheter 42 may implant the implantable member 46 in a different path surrounding the pin 94 defined by advancement of the catheter beyond the second opening of the tube.

It is also contemplated that the tube 92 may be embedded in an outer portion of the pin 94 thereby minimizing bulging of the pin. In yet another embodiment, an end of the introducer member 40 substantially drills through the outer surface of the broken bone 90 and the catheter 42 is deployed through the opening of the introducer member along a path defined by advancement of the catheter. The path of the catheter 42 may be substantially circumferential about the pin 94 which has previously been implanted into an inner portion of the broken bone 90. The implantable member 46 may be deposited from the catheter 42 in the substantially circumferential path as the catheter is retracted through the opening of the introducer member 40 as described above (see description of FIGS. 5A, B and C). The blocker, plunger or stylet 44 of the implantation device 200 may be utilized to prevent retraction of the implantable member 46 from the inner portion of the broken bone 90 of the body when the catheter 42 is retracted into the introducer member 40 after implanting the implantable member. It is understood that the circumferential path may have the shape of a spiral or any other suitable shape depending on the need to medicate a specific region of the broken bone 90. At least a portion of the implantation device 200 may have at least one marker to permit visualization of the location of the catheter 42 in the broken bone 90 of the body by at least one technique selected from the group comprising fluoroscopic x-ray, x-ray, computed tomography, magnetic resonance imaging and positron emission tomography (similar to the above description of FIG. 5A). In other embodiment any other technique may be utilized.

Exemplary Method of Distributing Therapeutic Material into a Body

In one example method of use or operation, the therapeutic material distribution method may comprise several steps. In one step of the method an implantation device 200 is provided comprising an introducer member 40 having an opening, a substantially flexible catheter 42 of the introducer member, the catheter being located in an inner portion of the introducer member and the catheter selectively extending from the inner portion of the introducer member through the opening of the introducer member. Furthermore, an implantable member 46 of the catheter 42 is guided along a path of an inner portion of a body receiving treatment. The implantable member is implanted into the inner portion of the body. The treatment material may be forced out of the implantable member or the implantable member may remain in the body. Retraction of the implantable member 46 from the inner portion of the body may be prevented with a blocker portion 44, which may comprise any element.

In greater detail, the method may comprise the steps of inserting the catheter 42 into a predetermined portion of the body and then advancing the catheter at a first programmable rate into an inner portion of the body along any path selected by a person or machine. Thereafter, the method may comprise extruding tissue material from the path of the body thereby providing the path having a channel defined by the advancement of the catheter 42. At this stage the catheter 42 may be retracted. This has the effect of leaving the implantable member 46 in the channel of the path. A blocker portion 44 may prevent the implantable material from being withdrawn with the catheter 42. It is understood that the catheter 42 may comprise a multiplicity of features to permit various activities including sampling, localizing and introducing of various implantable members 46.

Many areas of the body may be treated including, but not limited to, intervertebral disc 100, a breast tissue 60, a skeletal bone 90, a portion of the alimentary canal 80, a portion of the brain and nervous system 70, a portion of the circulatory system 80 and a portion of the respiratory system 70.

In one particular method of operation, the implantable member 46 is inserted in a substantially circumferential path of an inner portion of the body to facilitate at least one of measurement, localization, selection and sampling of body organ tissue material from an inner portion of the body. In an embodiment of the method performed on an intervertebral disc 100 the method comprises the step of advancing the catheter 42 into the transition zone 108 located between the annulus fibrosis 102 and the nucleus pulposus 104 of the intervertebral disc 100, and depositing any treatment material, which may comprise medication, stem cell infusion, or both and the like into the transition zone 108. In a further step of the method a portion of the implantation device 200 has at least one marker 48. In such a method, a person or machine may visualize the location of at least one marker 48 in the body. Various techniques to determine the location may comprise, but are not limited to fluoroscopic x-ray, x-ray, computed tomography, magnetic resonance imaging and positron emission tomography.

As discussed above, the method of operation may further comprise the step of injecting or extruding any treatment material, medication, stem cell infusion, or both from the catheter 42 of the implantation device 200 into a substantially circumferential path or any other path of an inner portion of the body. It is understood that the term “extruding” may also encompass various other methods of introducing or releasing, chemicals, medication, tissue or stem cell infusion or transplant and the like from the implantable member 46 depending on the physical form of the medication, stem cell infusion and the like. In and as discussed above, the implantable member 46 may comprise at least one material selected from the group consisting of a fluid, a slurry, a semi-solid, a gel, a matrix and a solid or be a structure configured to inject treatment material over a wide range of area and/or at a controlled rate. Where the fluid is substantially gaseous (such as an aerosol) simple flowing rather than “extruding” may result in proper dissemination of the medication.

While the invention has been particularly shown and described with reference to preferred embodiments thereof, it will be understood by those skilled in the art that the foregoing and other changes in form and details may be made therein without departing from the spirit and scope of the invention.

What is claimed is:

1. An intervertebral disc implantation device comprising, in combination:
(a) an introducer member having an opening;
(b) a substantially flexible catheter, said catheter located in an inner portion of said introducer member and said catheter configured to selectively extend from said inner portion of said introducer member through said opening of said introducer member;
(c) said catheter configured to both contain and selectively deliver an implantable member, said implantable member guided by said catheter along a path of an inner portion of said intervertebral disc of a person, said path located in a transition zone between a nucleus pulposus and an annulus fibrosis of said intervertebral disc; and
(d) a portion of said intervertebral disc implantation device preventing retraction of said implantable member from said inner portion of said intervertebral disc when said catheter is retracted into said introducer member after implanting said implantable member.

2. The device of claim 1 wherein said path has a substantially circumferential shape and, said implantable member is substantially located in a substantially circumferentially shaped portion of said transition zone or nucleus pulposis.

3. The device of claim 1 wherein said implantable member facilitates at least one of the following: measurement, localization, selection and sampling of material from said transition zone of said intervertebral disc.

4. The device of claim 1 wherein a wall of said circumferential implantable member comprises at least one of a diffusion membrane, a frangible wall portion and a wall portion having at least one perforation to permit placement of at least one treatment material from said implantable member into said transition zone of said intervertebral disc.

5. The device of claim 1 wherein said implantable member distributes at least one material from said implantable member to said transition zone thereby providing at least one localized infusion to nondestructively heal said intervertebral disc.

6. The device of claim 1 wherein said implantable member distributes at least one material from said implantable member to said transition zone thereby providing at least one continuous localized infusion to nondestructively heal said intervertebral disc.

7. The device of claim 1 wherein said implantable member comprises at least one material selected from the group consisting of a fluid, a slurry, a semi-solid, a gel, a matrix and a solid.

8. The device of claim 7 wherein said material of said implantable member comprises treatment material and the catheter is guidable.

9. An implantation device comprising, in combination:
an introducer member having an opening;
a substantially flexible catheter of said implantation device, said catheter located in an inner portion of said introducer member and said catheter selectively extending from said inner portion of said introducer member through said opening of said introducer member;
said catheter configured to both contain and selectively deliver an implantable member, said implantable member guided by said catheter along a path of an inner portion of a body receiving treatment; and
a portion of said implantation device preventing retraction of said implantable member from said inner portion of said body when said catheter is retracted into said introducer member after implanting said implantable member.

10. The device of claim 9 wherein an aspect of said body receiving treatment is selected from the group consisting of an intervertebral disc, a breast tissue, a skeletal bone, a portion of the alimentary canal, a portion of the brain and nervous system, a portion of the circulatory system and a portion of the respiratory system.

11. The device of claim 9 wherein said path has a substantially circumferential shape and, said implantable member is substantially located in a substantially circumferentially shaped inner portion of said body.

12. The device of claim 9 wherein said implantable member facilitates at least one of measurement, localization, selection and sampling of body organ tissue material from an inner portion of said body.

13. The device of claim 9 wherein a wall of said implantable member comprises at least one of a diffusion membrane, a self-dissolving membrane, a frangible wall portion and a wall portion having at least one perforation to permit placement of at least one treatment material from said implantable member into an inner portion of said body.

14. The device of claim 9 wherein said implantable member distributes at least one material from said implantable member to an inner portion of said body thereby providing at least one localized infusion to nondestructively heal said body.

15. The device of claim 9 wherein said implantable member distributes at least one material from said implantable member to an inner portion of said body thereby providing a continuous localized infusion to nondestructively heal said body.

16. The device of claim 9 wherein said implantable member comprises medication, stem cell infusion, or both.

17. The device of claim 9 wherein said catheter is inserted percutaneously into an inner portion of said body thereby causing minimal destruction of surrounding body tissue.

18. The device of claim 9 wherein at least a portion of said implantation device having at least one marker to permit visualization of the location of said catheter in said body by at least one technique selected from the group consisting of fluoroscopic x-ray, x-ray, computed tomography, magnetic resonance imaging and positron emission tomography.

19. The device of claim 9 wherein said implantable member comprises at least one material selected from the group consisting of a fluid, a slurry, a semi-solid, a gel, a matrix and a solid.

20. The device of claim 9 wherein said implantable member provides stinting of an inner portion of said body receiving treatment to both support and medicate said inner portion of said body.

21. A therapeutic material distribution method comprising the steps of:
providing an implantation device comprising an introducer member having an opening, a substantially flexible catheter of said introducer member, said catheter located in an inner portion of said introducer member and said catheter selectively extending from said inner portion of said introducer member through said opening of said introducer member;
guiding an implantable member of said catheter along a path of an inner portion of a body receiving treatment;
implanting said implantable member into said inner portion of said body; and
preventing retraction of said implantable member from said inner portion of said body with a portion of said implantation device while retracting said catheter into said introducer member after implanting said implantable member.

22. The method of claim 21 further comprising the steps of:
inserting said catheter into a predetermined portion of said body;
advancing said catheter at a first programmable rate into an inner portion of said body along said path;
extruding tissue material from said path of said body thereby providing said path having a channel defined by the advancement of said catheter;
retracting said catheter at a second programmable rate along said predetermined path; and
extruding said implantable member into said channel of said path while retracting said catheter.

23. The method of claim 21 further comprising the steps of:
providing an aspect of said body is selected from the group consisting of an intervertebral disc, a breast tissue, a skeletal bone, a portion of the alimentary canal, a portion of the brain and nervous system, a portion of the circulatory system, a portion of the respiratory system, cavity, canal, mass, and space occupying legion; and
implanting said implantable member in a substantially circumferential path or spiral path of an inner portion of said body to facilitate at least one of measurement, localization, selection and sampling of body organ tissue material from an inner portion of said body.

24. The method of claim 21 further comprising the steps of:
providing at least a portion of said implantation device having at least one marker;
visualizing the location of said at least one marker in said body by at least one technique selected from the group consisting of fluoroscopic x-ray, x-ray, computed tomography, magnetic resonance imaging and positron emission tomography; and
extruding medication, stem cell infusion, or both from said catheter of said implantation device into a substantially circumferential path of an inner portion of said body.

25. An device configured to apply electrical, magnetic, or chemical energy to a vertibre disc comprising:
an introducer member having an opening;
a substantially flexible catheter of said implantation device, said catheter located in an inner portion of said introducer member and said catheter selectively extending from said inner portion of said introducer member through said opening of said introducer member;
said catheter configured to apply or insert into the disc an apparatus that may apply electrical, magnetic, or chemical energy to the disc, wherein apply electrical, magnetic, or chemical energy to the disc selectively stimulates or promotes intra discal function or delivery of material into our out of the disc.

26. The device of claim 25, wherein the chemicals comprise antibiotics.

27. The device of claim 25, the device is configured to pump material into or out of the disc in connection with disc compression and expansion.
